(19) United States
(12) Patent Application Publication
(10) Pub. No.: US 2011/0052661 A1
(43) Pub. Date: Mar. 3, 2011

(54) PET DENTAL HYGIENE PRODUCT

(76) Inventor: Susan Datz Weiss, Bonita Springs, FL (US)

(21) Appl. No.: 12/867,529

(22) PCT Filed: Feb. 13, 2009
(86) PCT No.: PCT/US09/34029

§ 371 Date: Nov. 10, 2010

Related U.S. Application Data
(60) Provisional application No. 61/028,407, filed on Feb. 13, 2008.

(51) Int. Cl. A61K 9/00; A61K 38/44; A61K 33/42; A61K 31/34; A61K 31/047; A61P 31/04; A61P 1/02

(52) U.S. Cl. 424/442; 424/94.4; 424/605; 514/474; 514/738

(57) ABSTRACT

An edible product for oral hygiene of a pet, the product being chewable by the pet to remove plaque, tartar and stain from the teeth of the pet and to reduce breath odor of the pet. The dental hygiene product includes a body formed of a first set of constituent components and a second set of constituent components, the first set of constituent components including abrasive components and one or more breath freshening components, the combination when chewed by the pet effecting the removal of plaque, tartar and stain and the inhibition of breath odor, the second set of constituent components including an abrasive component and one or more active components, the abrasive component when chewed by the pet effecting the removal of plaque, tartar and stain from the teeth of the pet, the one or more active components being characterized by the active components disrupting growth of bacteria in the pet's mouth thereby inhibiting plaque and tartar formation and breath odor from a bacterial source.

The present application claims priority to U.S. Provisional Application No. 61/028,407 filed on Feb. 13, 2008 and entitled PET DENTAL CHEW, the entire contents of which are herein incorporated by reference.

### FIELD OF THE INVENTION

The present invention general related to products for the oral care of pets and to methods of providing oral pet care. More particularly, the invention relates to a dental hygiene product that combats plaque and tartar formation, while also providing breath freshening for pets, namely dogs and cats.

### BACKGROUND OF THE INVENTION

Pets, such as dogs and cats, are susceptible to a variety of dental health issues. Some of these issues include bad breath, plaque and tarter formation, gum irritation and stained teeth. The source of many of these problems originates in the chemistry of the pet's mouth, and in particular, in its saliva.

Plaque is a sticky film, consisting of bacteria and other components, that becomes very tightly adhered to the teeth. Once adhered, it is not easily washed away. Tartar, also known as dental calculus, results from the thickening and hardening (mineralization) of plaque. As tartar develops, it becomes visibly white or yellowish in color and may become stained by foods or other products eaten or chewed by the pet.

While being undesirable from a purely aesthetic standpoint, tartar build-ups are also a source of irritation in the pet's mouth and are a contributing factor in gingivitis. Once formed, tartar is virtually impossible to remove, except by mechanical measures, the most common form of which is abrasively scraping the tartar off of the pet's teeth.

Plaque bacteria are also believed to produce acids that eat away the enamel of the teeth. This in turn results in tooth decay of and cavities. Tooth decay begins at the surface of the tooth and may eventually progress through the dentine of the tooth and into the pulp cavity. Once in the pulp cavity, this decay may result in loss of the tooth.

The bacteria in a pet's mouth are also a significant cause of bad breath. A wide variety of chemical and biological agents have been suggested to slow or prevent plaque and tarter formation or to remove plaque after it has formed. In humans, prevention and removal of this material is routinely done by brushing, flossing, using mouth washes and by mechanical removal (scraping and polishing) at a dentist's office. When it comes to pets, however, the pet owner, while being well intentioned, usually only briefly adheres to a routine of dental hygiene for their pets. Compounding this, many pets find brushing and flossing by their owners to be an unpleasant experience and, therefore, are uncooperative during the process.

Since pet foods do not provide sufficient cleaning to remove plaque (pet food is typically consumed very rapidly with little chewing on the part of the pet), a wide variety of chew products have been manufactured for pets. These chew products are intended to provide a mechanically abrasive action that will remove the plaque and tartar, while also massaging the gums of the pet. Such products include pieces of rawhide (in various forms and shapes), rope (knotted on its ends to resemble bones and other structures), as well as biscuits and treats having a formulation that requires additional chewing on the part of the pet and thereby results in additional abrasive action to remove the plaque. In addition, each of these products may be provided with chemical or biological agents that also operate to prevent plaque build up or bad breath.

With the present invention, an alternative product and method for reducing or alleviating the above dental problems is provided.

### SUMMARY OF THE INVENTION

In view of the above noted limitations and drawbacks of the known technology, the present invention provides an edible and chewable product for oral hygiene of a pet. The product is chewed by the pet to remove plaque, tartar and stain from the teeth of the pet and to reduce breath odor of the pet.

More specifically, the product includes a body that is formed with first and second sets of constituent components. The first set of constituent components includes a combination of abrasive components and one or more breath freshening components. The combination, when chewed by the pet, effects the removal of plaque, tartar and stain and inhibits the formation of breath odor. The second set of constituent components includes abrasive components and one or more active components. The abrasive components, when chewed by the pet, cause removal of plaque, tartar and stain from the teeth of the pet, while the active components disrupt the growth of bacteria in the pet's mouth and thereby inhibit plaque and tartar formation and breath odor from a bacterial source.

In one aspect of the invention, the first and second sets of constituent components are provided heterogeneously throughout the body of the product.

In another aspect of the invention, the first and second set of constituent components is provided in distinct portions of the body of the product.

In a further aspect, the first set of constituent components is provided in a first layer of the body and the second set of constituent components is provided in a second layer of the body.

In yet another aspect of the invention, the first layer is an outer layer of the body and the second layer is an inner layer of the body, the inner layer being at least partially surrounded by the outer layer.

In still another aspect of the invention, the first set of constituent components is a composition based on one or more ingredients selected from the group of rice, corn, oats, oat flour, wheat, wheat gluten, hydrolyzed wheat protein, wheat protein isolate, soy, hydrolyzed soy protein, soy protein isolate, potatoes, potato flour, or derivative thereof.

In a further aspect of the invention, the second set of constituent components includes one or more of glucoxidase, sodium pyrophosphate, an ascorbic acid compound, sorbitol, and lactoperooxidase.

It is also an aspect of the invention to provide a dental hygiene product for a pet, the product being chewable by the pet to remove plaque, tartar and stain from the teeth of the pet and to reduce breath odor of the pet. The dental hygiene product comprising an edible body having a first layer and a second layer. The first layer includes a combination of abrasive components and one or more breath freshening components, wherein the combination, when chewed by the pet, effects the removal of plaque, tartar and stain and the inhibition of breath odor. The second layer includes a toothpaste material having an abrasive component and one or more active components. When chewed by the pet, the abrasive component causes the removal of plaque, tartar and stain from the teeth of the pet, while the one or more active components cause a disruption in the growth of bacteria in the pet's mouth, thereby inhibiting plaque and tartar formation and breath odor from a bacterial source.

In a further aspect, the body of the product is an unbaked chewable product.

In another aspect, the first layer forms an outer layer of the product.

In still another aspect, the second layer forms an inner layer of the product.

In yet another aspect, the second layer is encircled by the first layer.

It is also an aspect of the invention that body defines a length having opposing ends, with the first and second layers being visible at the ends of the body.

In a further aspect, the first and second layers are different in color from one another.

In another aspect, the second layer defines a cross sectional shape comprising one of a geometric shape and a symbol.

In yet another aspect, the body defines an outer surface having a series of protrusions extending therefrom.

In still another aspect, the body defines a length and the protrusions extend the length of the body.

It is also an aspect of the invention that the first layer includes a composition based on one or more ingredients selected from the group of rice, corn, oats, oat flour, wheat, wheat gluten, hydrolyzed wheat protein, wheat protein isolate, soy, hydrolyzed soy protein, soy protein isolate, potatoes, potato flour, or derivative thereof.

It is also an aspect of the invention that the active components of the second layer include one or more of glucoxidase, sodium pyrophosphate, an ascorbic acid compound, sorbitol, and lactoperooxidase.

In a further aspect, the invention provides a method of preventing and/or reducing breath odor and plaque and tartar buildup in a pet's mouth, the method comprising the steps of: contacting the teeth of the pet with a dental hygiene product having a first set of constituent components and a second set of constituent components; the first set including a combination of abrasive components and one or more breath freshening components and effecting the removal of plaque, tartar and stain and inhibiting breath odor by chewing of the dental hygiene product; the second set including a toothpaste material with an abrasive component and one or more active components, effecting the removal of plaque, tartar and stain from the teeth of the pet and disrupting growth of bacteria in the pet's mouth thereby inhibiting plaque and tartar formation and breath odor from a bacterial source by chewing of the dental hygiene product so as to engage the teeth of the pet with the abrasive component and the one or more active components.

### BRIEF DESCRIPTION OF THE DRAWINGS

FIG. 1 is a perspective view of an dental hygiene product for pets in accordance with the principles of the present invention; and

FIGS. 2a-2f are transverse, cross sectional views through various embodiments of dental hygiene products, similar to that seen in FIG. 1, embodying the principles of the present invention.

### DETAILED DESCRIPTION

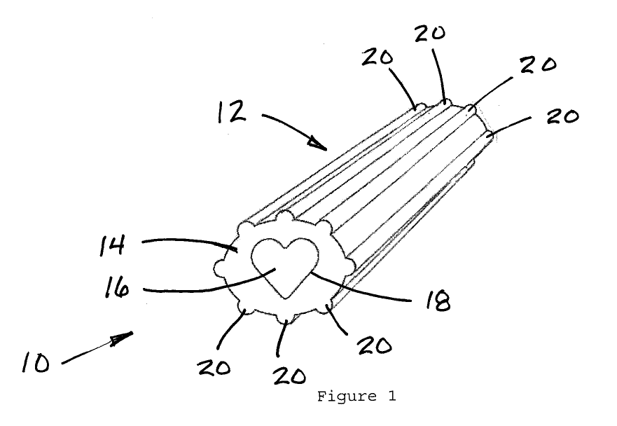

Referring now to the figures, the present invention provides a pet dental hygiene product, such as a dental chew, that operates on the bacteria in a pet's mouth to reduce or prevent the development of plaque and tartar and the problems associated therewith. Also, the product reduces or eliminates bad breath caused by the bacteria. In addition to combating breath odors on the bacterial level, the dental hygiene product further includes a breath freshener to aid in masking odors that have not been eliminated. One embodiment of the product is seen in FIG. 1 and is generally designated at 10.

As represented in FIG. 1, the product 10 is a dental hygiene product having a body 12 provided in an elongated form and comprised of an outer layer 14 and an inner layer 16. The outer and inner layers 14, 16 each provide specific attributes for combating the above noted oral health problems. The outer layer 14 is generally constructed to provide breath freshening benefits while also providing mechanical action for removing plaque and tartar through abrasion during chewing. The inner layer 16 includes a toothpaste type of product to control plaque, tartar and bacterial caused by bad breath. This is achieved by disrupting the formation of plaque and the growth of bacteria.

More specifically, the outer layer 14 of the product 10 includes a combination of breath freshening components and abrasive components. Preferably, the breath freshening components of the outer layer 14 include one or more of chlorophyll, cinnamon and clove (or similar constituents), each of which reduces breath odors in pets. The abrasive components, as well as binders and/or fillers, make up the remaining portion of the outer layer 14. Preferably, the outer layer 14 is of a rice-based composition. However, the base composition of the outer layer 14 may alternatively include one or more of the following ingredients: wheat, wheat gluten, hydrolyzed wheat protein, wheat protein isolate, hydrolyzed soy protein, soy protein isolate, soy, corn, oats, oat flour, potatoes, potato flour, or derivatives thereof.

The inner layer 16 is a toothpaste-type of material and includes, not only a cleansing/abrasive component, but also one or more active components that disrupt the growth of bacteria in the pet's mouth. By reducing bacteria in the pet's mouth, plaque and tartar formation are inhibited or reduced, as is the growth of oral germs causing bad breath.

Preferably, the inner layer 16 includes one or more of sodium bicarbonate (baking soda), calcium carbonate, sorbitol, lactoperooxidase or similar constituents. These constituents operate as cleansing agents and provide abrasive action and scrubbing power to the inner layer.

The composition of the inner layer 16 also preferably includes an effective amount of one or more of glucoxidase, sodium pyrophosphate, an ascorbic acid compound, sorbitol, lactoperooxidase, or similar constituents, as active components that kill or inhibit bacteria growth.

Glucoxidase is a microbial enzyme that reacts with oxygen. By reacting with oxygen, this enzyme operates to deprive aerobic bacteria of the oxygen they need for survival.

Sodium pyrophosphate is a tartar control agent and operates to remove calcium and magnesium from the pet's saliva. By removing calcium and magnesium, these minerals are prevented from being deposited on teeth and providing a medium for bacteria growth.

While the ascorbic acid compound is preferably a compound with at least 45% ascorbic acid, lesser amounts may be utilized, although with possibly lesser effectiveness. The ascorbic acid compound not only disrupts plaque and tartar formation, it also slows the growth of oral bacteria. By slowing the growth of oral bacteria, the bacterial source of bad breath odors is inhibited or reduced. One available source of a suitable ascorbic acid compound is available from DS Nutritional Products (Parsippany, N.J.) under the brand names of Stay-C™ 50 and Stay-Clean™.

Sorbitol is a compound that aids in reducing the demineralizing potential, and therefore development, of plaque.

Lactoperooxidase is a peroxidase enzyme that has antimicrobial and antioxidant properties.

The remainder of the inner layer 16 may be comprised of various well known binders and/or fillers that are compatible with the above constituents.

The dental hygiene product 10 is formed by mixing the desired ones of the inner layer constituents together and by mixing the desired ones of the outer layer constituents together, and then co-extruding the inner layer 16 within the outer layer 14. The extrudate is then cut to length to form the product 10 in the form of a dental hygiene product. The actual length of the dental hygiene product is one of design choice and will be generally determined by the size and type of pet for which it is intended. The overall or effective diameter of the dental hygiene product can also vary depending on the particular size and type of pet for which it is intended. While not intending to be bound by any numerical range, it is preferred that the effective outer diameter for the body of the dental hygiene product will be in the range of about 0.75-1.5 inches.

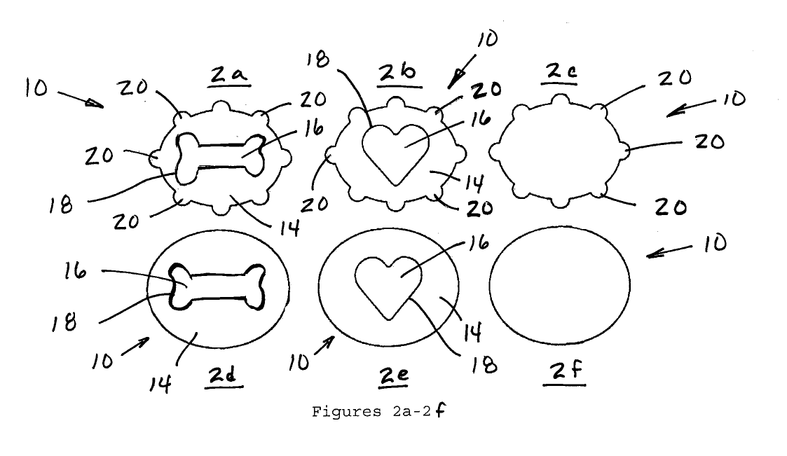

Additionally, the cross-sectional shape of the body 12 of the dental hygiene product may be practically any desired shape, including, without limitation, round, circular, ovoid, and rectangular. The inner layer 16 may be of the same or different color than the outer layer 14. As such, the boundary 18 between the inner layer 16 and the outer layer 14 may be distinguishable or indistinguishable. Additionally, this boundary may define the same general shape as the cross sectional shape of the dental hygiene product or it may define a different decorative shape, such as a heart (illustrated), star, bone, diamond or other geometric shape. Alternatively, the exterior surface of the body 12 of the dental hygiene product may be smooth along its length or provide protrusions 20 that enhance the effectiveness of the scrubbing of the pet's teeth. As seen in FIGS. 1 and 2a-2c, the exterior surface of the dental hygiene product is provided with a series of protrusions 20 that form ridges along its length. In an alternative embodiment, the protrusions 20 may be formed intermittently about the body 12 of the dental hygiene product. A smooth exterior surface is seen in the embodiments of FIGS. 2d-2f.

For the various constituents of the dental hygiene product, it is preferred that a variety of all natural constituents are used. However, non-all natural constituents could be used in the dental hygiene product without impacting the effectiveness of the product.

As thus far described, the present invention includes separately identified outer and inner layers 14, 16. In a further embodiment, the previously described constituents of the outer and inner layers 14, 16 are combined to form a product of uniform consistency throughout. Thus, the product is of a single layer or monolithic construction. Such a construction is seen in FIGS. 2c and 2f, of which FIG. 2c illustrates the product 10 with protrusions 20 located about its exterior surface and FIG. 2f illustrates the product with a smooth exterior surface.

As a further embodiment, the constituents of the inner layer 16 could alternatively be on the exterior of the product 10. As such, the product 10 would be formed without a distinguishing inner layer and the constituents of the inner layer applied to the exterior surface of the product 10 and thereby incorporated into the product 10. For example, those constituents may be applied to the exterior surface of the product 10 by a method similar to coating or basting, resulting in those constituents forming a coating on the exterior surface of the product or being absorbed into the product through the exterior surface.

As so far described, the present invention has been incorporated into a dental hygiene type of product. As will be appreciated, the form factor in which the invention is provided can vary from an elongated dental hygiene product. It is contemplated that the invention may be provided in a form whereby the constituents of the previously described inner layer are carried and delivered to the pet in a chewable capsule, tablet, chewable, chewing gum, gel or spray form.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

1. An edible product for oral hygiene of a pet, the product being chewable by the pet to remove plaque, tartar and stain from the teeth of the pet and to reduce breath odor of the pet, the product comprising:
a body formed of a first set of constituent components and a second set of constituent components, the first set of constituent components provided as an outer layer of the body, the second set of constituent components being provided as an inner layer of the body at least partially surrounded by the outer layer, the first set of constituent components including abrasive components and one or more breath freshening components, the combination when chewed by the pet effecting the removal of plaque, tartar and stain and the inhibition of breath odor, the second set of constituent components including an abrasive component and one or more active components, the abrasive component when chewed by the pet effecting the removal of plaque, tartar and stain from the teeth of the pet, the one or more active components being characterized by the active components disrupting growth of bacteria in the pet's mouth thereby inhibiting plaque and tartar formation and breath odor from a bacterial source.

2-5. (canceled)

6. The edible product of claim 1 wherein the first set of constituent components is a composition based on one or more ingredients selected from the group of rice, corn, oats, oat flour, wheat, wheat gluten, hydrolyzed wheat protein, wheat protein isolate, soy, hydrolyzed soy protein, soy protein isolate, potatoes, potato flour, or derivative thereof.

7. The edible product of claim 1 wherein the second set of constituent components includes one or more of glucoxidase, sodium pyrophosphate, an ascorbic acid compound, sorbitol, and lactoperooxidase.

8. The dental hygiene product of claim 1 wherein the body is an unbaked chewable product.

9. The dental hygiene product of claim 1 wherein the body is an extrudate.

10. The dental hygiene product of claim 1 wherein the first set of constituents is provided in an outer layer.

11. The dental hygiene product of claim 1 wherein the second set of constituents is provided in an inner layer.

12. The dental hygiene product of claim 11 wherein the inner layer is encircled by the outer layer.

13. The dental hygiene product of claim 12 wherein the body defines a length having opposing ends, the first and second layers being visible at the ends of the body.

14. The dental hygiene product of claim 13 wherein the first and second layers are different in color from one another.

15. The dental hygiene product of claim 13 wherein the second layer defines a cross sectional shape comprising one of a geometric shape or a symbol.

16. The dental hygiene product of claim 1 wherein the body defines an outer surface having a series of protrusions extending therefrom.

17. The dental hygiene product of claim 16 wherein the body defines a length and the protrusions extend the length of the body.

18. A method of preventing and/or reducing breath odor and plaque and tartar buildup in a pet's mouth, the method comprising the steps of:
contacting the teeth of the pet with a dental hygiene product having a body including a first set of constituent components and a second set of constituent components, the first set of constituent components provided as an outer layer of the body, the second set of constituent components being provided as an inner layer of the body at least partially surrounded by the outer layer;
the first set of constituent components including a combination of abrasive components and one or more breath freshening components and effecting the removal of plaque, tartar and stain and inhibiting breath odor by chewing of the dental hygiene product;
the second set of constituent components including a toothpaste material with an abrasive component and one or more active components, effecting the removal of plaque, tartar and stain from the teeth of the pet and disrupting growth of bacteria in the pet's mouth thereby inhibiting plaque and tartar formation and breath odor from a bacterial source by chewing of the dental hygiene product so as to engage the teeth of the pet with the abrasive component and the one or more active components.

19. The method of claim 18 wherein the active components of the second layer include one or more of glucoxidase, sodium pyrophosphate, an ascorbic acid compound, sorbitol, and lactoperooxidase.

20. The method of claim 18 wherein first layer includes a composition based on one or more ingredients selected from the group of rice, corn, oats, oat flour, wheat, wheat gluten, hydrolyzed wheat protein, wheat protein isolate, soy, hydrolyzed soy protein, soy protein isolate, potatoes, potato flour, or derivative thereof.

21. (canceled)
